(19) United States
(12) Patent Application Publication
(10) Pub. No.: US 2009/0005794 A1
(43) Pub. Date: Jan. 1, 2009

(54) Suture Holding Device

(75) Inventor: Kent J. Lowry, Rhinelander, WI (US)

Correspondence Address: HARNESS, DICKEY & PIERCE, P.L.C., P.O. BOX 828, BLOOMFIELD HILLS, MI, 48303, US

(73) Assignee: BIOMET SPORTS MEDICINE, INC., Warsaw, IN (US)

(21) Appl. No.: 11/770,500

(22) Filed: Jun. 28, 2007

(51) Int. Cl. A61B 17/04

(52) U.S. Cl. 606/148

(57) ABSTRACT

A flexible member managing device is provided. The flexible member managing device includes a biocompatible body having a base, an extension projecting from the base, and a plurality of flexible member-receiving slots to organize a plurality of flexible members. The flexible member managing device can be repeatedly fixed to allow movement and temporary fixation of the flexible member managing device intraoperatively.

### FIELD

The present disclosure relates to apparatus and methods for securing a flexible member.

### BACKGROUND

The statements in this section merely provide background information related to the present disclosure and may not constitute prior art.

During certain surgical procedures, the surgeon must manage several pieces of suture or flexible strands. Common suture management techniques include placing a grasper around the suture to crimp the suture and then letting the graspers hang or otherwise rest at the surgical site. The sutures often become tangled and additional surgical time is used to organize and verify the location of particular sutures. For example, with rotator cuff repair, it is not uncommon to use six or more sutures at the surgical site. These six or more sutures are often coming from a cannula, thereby reducing the organization of the sutures, or the sutures can come from an open surgical site. Accordingly, there is a need to provide a suture management system that is efficient and allows for quick identification and separation of sutures.

### SUMMARY

The present teachings provide methods of managing a plurality of flexible members at a surgical site. At least one flexible member is aligned in at least one of a plurality of flexible slots defined by a flexible member managing device to organize the plurality of flexible members. The flexible member managing device is then arranged remote from the surgical site.

The present teachings also provide methods of managing a plurality of flexible members at a surgical site. At least one of the plurality of flexible members is attached to a rotator cuff. The flexible members are then aligned with at least one of the plurality of flexible member receiving slots defined by the flexible member managing device to organize the flexible members. The flexible member managing device is then arranged remote from the rotator cuff thereby tensioning the flexible member and effecting tissue closure of the rotator cuff.

The present teachings also provide a flexible member managing device. The flexible member managing device comprises a biocompatible body having a base and an extension from the base. At least a portion of the extension defines a plurality of flexible member receiving slots to organize a plurality of flexible members. An attachment region on the flexible member managing device provides repeated, removable fixation of the flexible member managing device.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIGS. 1A-1E depict various flexible member managing devices according to various embodiments;

FIG. 2 depicts a bottom view of a flexible member managing device according to various embodiments;

FIG. 3 depicts a loaded flexible member managing device according to various embodiments;

FIG. 4 depicts a flexible member managing device at a surgical site according to various embodiments;

FIG. 5 depicts a flexible member managing device used with a suture anchor according to various embodiments;

FIG. 6 depicts a top-view of a refractor and flexible member managing device combination according to various embodiments;

FIG. 7 depicts an exploded side-view of a retractor and flexible member managing device combination according to various embodiments;

FIG. 8 depicts a perspective view of a retractor and flexible member managing device combination according to various embodiments; and

FIG. 9 depicts a retractor and flexible member managing device combination at a surgical site according to various embodiments.

### DETAILED DESCRIPTION

The following description is merely exemplary in nature and is not intended to limit the present disclosure, application, or uses.

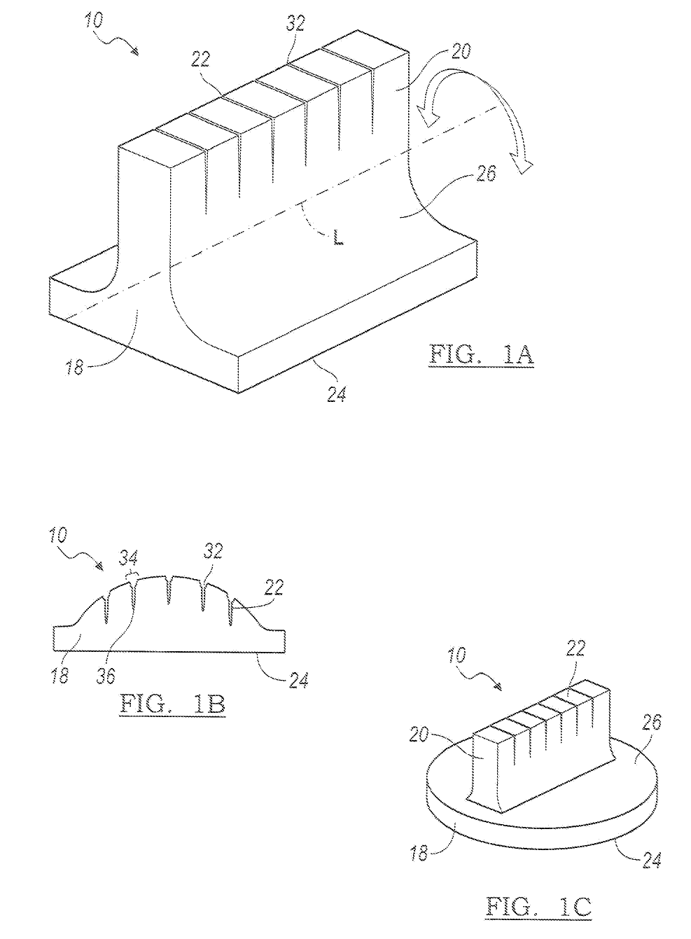

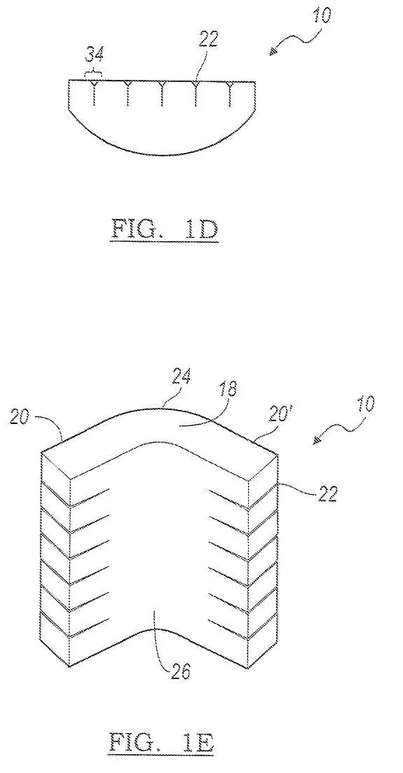

Referring to FIGS. 1A through 9, a flexible member managing device 10 is provided. The flexible member managing device 10 can be used to manage, organize, or provide tension on a flexible member 12 during a surgical procedure. The flexible member managing device 10 is easy to use, provides for simple, intraoperative relocation of the device 10 at the surgical site without significant manipulation by the user, and is useful in a variety of surgical procedures.

While rotator cuff repair is described, it is understood that the devices and surgical techniques can easily be adapted for other orthopedic and non-orthopedic uses. The surgical procedures can be used to fix soft tissue, bone, cartilage, and the like. The surgical procedures can be used to fix or join similar tissue types (cartilage to cartilage) or dissimilar tissue types (bone to soft tissue). The surgical techniques can include minimally invasive and open surgical procedures.

The flexible member 12 can be made from any biocompatible material that is flexible and can pass through and secure a tissue. Exemplary materials include, but are not limited to, non-resorbable polymers, such as polyethylene or polyester, resorbable polymers, metals, and various combinations thereof. The materials can include those formed into a monofilament, multiple filaments, cables, wires, braids, and the like. In various embodiments, the flexible member 12 can be a hollow material. In various embodiments, the flexible member 12 can be a suture 14.

The flexible member managing device 10 includes a body 18 having a base 18 and an extension 20 projecting from the base 18. The extension 20 defines a plurality of flexible member receiving slots 22 to organize a plurality of flexible members 12.

The flexible member managing device 10 can be made of any biocompatible material. Exemplary materials include, but are not limited to silicone, rubber, and other biocompatible polymers; metals, such as titanium, stainless steel, cobalt, the like; and combinations thereof. The flexible member managing device 10 materials can be disposable materials or multiple-use materials. The flexible member managing device 10 can include resilient areas (such as silicone or rubber areas) that allow the flexible member 12 to be squeezed in the flexible member receiving slots 22 without damage to the flexible member 12. The resilient areas can provide both tight securing of the flexible member 12 and easy removal thereof. The flexible member managing device 10 can also be made of rigid and non-resilient materials or combinations of resilient and non-resilient materials.

The base 18 includes a lower surface 24 and an upper surface 26. The lower surface 24 can be planar surface, as shown in FIGS. 1A through 1C, or can be a curved surface as shown in FIGS. 1D and 1E. The lower surface 24 is the region by which the flexible member managing device 10 is placed at the surgical site 28 and is moved to provide tension on the flexible member 12, as disclosed later herein.

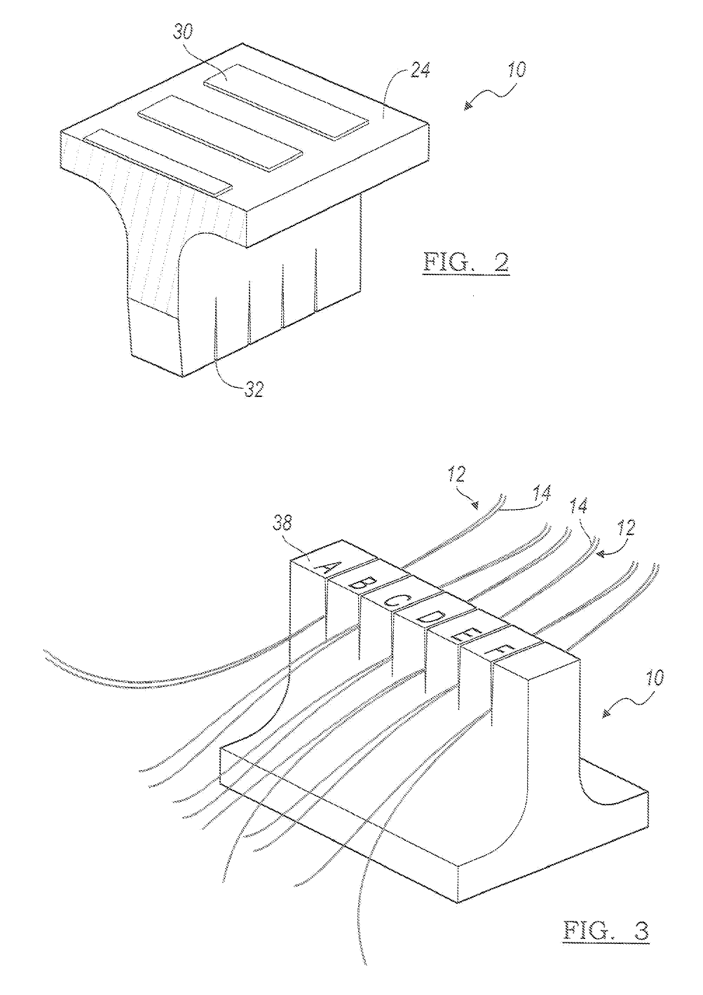

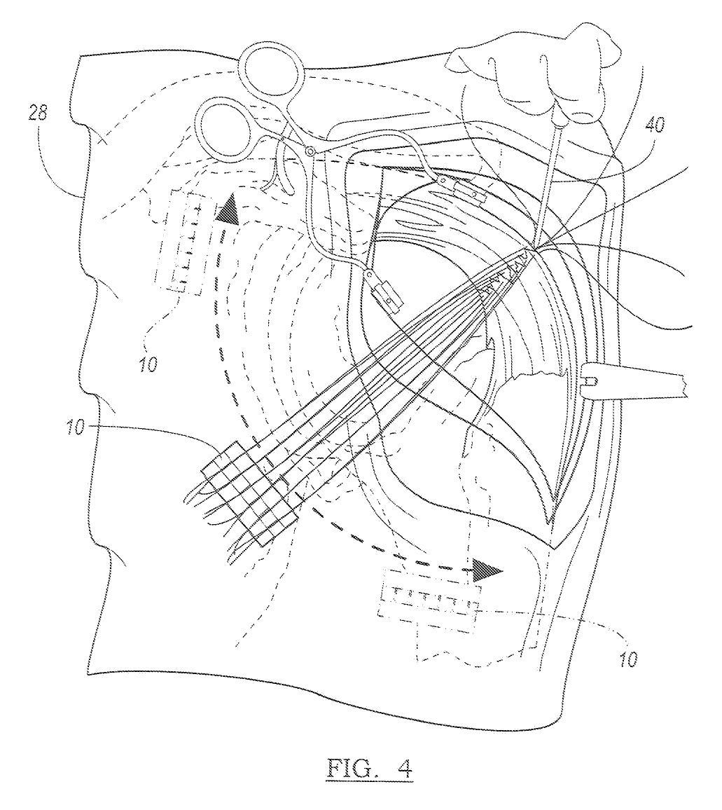

As shown in FIG. 2, the lower surface 24 can include an attachment in the form of an adhesive region 30 to temporarily secure the flexible member managing device 10 to the patient's skin or on a surface in the immediate vicinity of the surgical site, such as a gown on the patient, a surgical drape, a table near the patient, or the surgical table, as non-limiting examples. Other attachments are detailed later herein. The adhesive region can be a multiple-use adhesive such that the flexible member managing device 10 can be positioned and repositioned as needed. For example, the flexible member managing device 10 can be attached to a first location, easily removed from the first location, and then attached to a second location while the flexible members 12 are carried by the flexible member managing device 10. An example of movement of the flexible member managing device 10 is shown in FIG. 4. The range of motion can also include inclining the flexible member managing device 10 with respect to the surgical site 28 by lifting the flexible member managing device 10 from a position of rest. The range of motion can also include declining the flexible member managing device 10 with respect to the surgical site 28 by lowering the flexible member managing device 10 from a position of rest. It is understood, however, that the range of motion depicted in FIG. 4 is non-limiting and is for illustrative purposes only.

In various embodiments, the adhesive region 30 covers a region of the lower surface 24 using strips (shown in FIG. 2) or with adhesive dots or the like. In other embodiments, the adhesive region 30 can cover the entire lower surface 24. The adhesive region 30 can be removable from and/or replaceable on the lower surface 24. The adhesive region 30 can be pre-fixed on the base 18 or can be attached to the base 18 prior to use. The adhesive region 30 materials can be selected based on the type of material used for the flexible member managing device 10. In embodiments where the flexible member managing device 10 is made of a medical grade silicone polymer, the base 18 can serve as the adhesive region 30 due to the tacky or sticky properties of the medical grade silicone which cause it to attach to the skin.

The adhesive region 30 can utilize a mechanical adhesion, such as that from a suction cup, a frictional force between the adhesive region and the underlying surface, such as the frictional force provided when using a silicone pad, or a chemical adhesion, such as the temporary adhesion provided by a temporary surgical tape, biocompatible glue, or fixative. A fabric such as Velcro can also be used to secure the flexible member managing device 10. It is understood that various adhesive materials or techniques not listed herein are also contemplated as useful with the present teachings. The type of adhesive region 30 and the materials used are selected based on for example, the area to which the adhesive region will adhere. For example, the materials used will be different based on whether the adhesive region is going to contact the patient's skin, a covering on the patient, a surgical table under the patient, or a surface adjacent to the patient, such as a tray. The type of adhesive used will also be selected based on whether the particular surgical procedure requires that the flexible member managing device 10 be moved frequently.

Referring to FIGS. 1A through 1E, the upper surface 26 of the flexible member managing device 10 includes an extension 20 defining a plurality of flexible member receiving slots 22. The upper surface 26 can include a single extension 20 as shown in FIG. 1A or multiple extensions 20 and 20′ such as shown in FIG. 1E. The extension 20 can be perpendicular to the base or the extension 20 can be otherwise angled with respect to the base 18. As shown in FIG. 1E, the extensions 20 and 20′ have different angles with respect to the base 18. In various embodiments, the extension 20 and the base 18 can be formed as a single piece or can snap or otherwise fit together to form the flexible member managing device 10.

The flexible member receiving slots 22 extend partially through the extension 20. As shown in FIG. 1A, the flexible member receiving slots 22 extend through the extension 20 and towards the base 18 and are generally perpendicular to a horizontal axis of the base 18. As shown in FIG. 1E, the flexible member receiving slots 22 can have different angles towards the base 18 depending on the angle of the extensions 20 and 20′.

In various embodiments, the flexible member receiving slots 22 can be sized to grippingly engage the flexible members 12 to prevent unintentional dislodging of the flexible members 12, yet still allow for uncomplicated removal of the flexible members 12 by the user. The flexible member receiving slots 22 can include a smooth interior to protect the flexible member 12. The flexible member receiving slots 22 can also be sized to allow the flexible member 12 to rest in the flexible member receiving slot 22 without grippingly engaging the flexible member. For example, the flexible member 12 can loosely rest in the flexible member receiving slot 22.

Referring to FIGS. 1A and 1B, the flexible member receiving slots 22 are tapered or can include an opening 32 having a narrowed region in which to contain the flexible member 12. The openings 32 can be sized to fit a single flexible member 12, a folded flexible member 12, or a plurality of flexible members 12. As shown in FIG. 1A, the size of the opening 32 is a taper having a constantly decreasing space. As shown in FIG. 1B, the opening 32 is a taper with a non-constant size decrease and includes an upper funnel region 34 having a differently shaped and narrower taper 36 underneath. In such embodiments, the upper funnel region 34 guides the flexible member 12 into the bottom of the flexible member receiving slot 22. The flexible member 12 can also rest in the funnel region 34 and not advance to the bottom of the flexible member receiving slot 22. In still other embodiments, the flexible member receiving slots 22 can be curved or rounded.

As shown in FIG. 3, the flexible member receiving slots 22 can be identified using indicators 38. Suitable indicators 38 include letters, numbers, colors, shapes, or the like. The respective flexible members 12 can be placed in a marked flexible member receiving slot 22 and later identified and removed for use. The indicators 38 can also be used to replace the flexible member 12 in the appropriate slot 22 or to indicate that a particular flexible member 12 has been engaged with the tissue or need not be engaged with a certain region of the tissue.

Referring to FIGS. 3 through 5, the present teachings provide methods of managing flexible members 12 at the surgical site 28. First, the flexible member 12 is prepared for use to attach the tissue 40. In various embodiments, the flexible members 12 are first placed into tissue 40 and then aligned or placed in the flexible member managing device 10. In other embodiments, the flexible members 12 are first aligned or placed into the flexible member managing device 10 and then placed into the tissue 40.

To align the flexible member 12 in the flexible member receiving slot 22, at least one flexible member 12 is placed in an opening 32 on the flexible member receiving slot 22. As detailed above, multiple flexible members 12 can be placed in the flexible member receiving slots 22 and can be correlated to an indicator 38. The flexible member 12 can be folded over or singly placed in the flexible member receiving slot 22. Multiple flexible members 12 can also be placed in the receiving slots 22, such as shown in FIG. 3. Once the flexible member 12 is placed in the flexible member receiving slot 22 it can be held as to not be unintentionally dislodged or can loosely rest in the opening 32. Placing the flexible members 12 in the flexible member receiving slots 22 organizes the flexible members 12 and makes the surgical procedure less cumbersome and more efficient.

After the flexible member 12 is placed in the appropriate flexible member receiving slot 22, the flexible member managing device 10 can be moved a distance from the surgical site 39 to pull the flexible member 12 taut, as illustrated in FIG. 4. Further pulling or movement of the flexible member managing device 10 provides additional tension on the flexible member 12 to tighten the tissue 40, retract the tissue 40, or effect tissue closure. In various embodiments, engaging the flexible member managing device 10 is achieved by grasping the base 18 and retracting the device 10 away from the surgical site 28 or inclining or angling the device 10 with respect to the surgical site 28 to place tension on the flexible member 12 and thereby tighten the tissue 40. The tension is simultaneously placed on all flexible members 12 on the flexible member managing device 10. As stated above herein, the arrows in FIGS. 4 and 9 depict exemplary ranges of motion through which the flexible member managing device can be moved to provide tension on the suture and subsequently tighten the tissue 40.

In still other embodiments, the flexible members 12 can also be tensioned by leaning the extension 20 away from the surgical site 28. Leaning the extension 20 can provide the refined tightening of the tissue 40 without significantly displacing the flexible member managing device 10. Leaning the extension 20 can be achieved by rotating or angling the base 18 with respect to the longitudinal axis L as depicted in FIG. 1A.

The flexible members 12 remain in the respective flexible member receiving slot 22 throughout the movement or angling. The organized flexible members 12 on the flexible member managing device 10 provide even tension on the tissue 40 and can be used to simultaneously pull and tighten all of the flexible members 12.

The user can maintain tension on the flexible members 12 by keeping the flexible members 12 taut while they are in the flexible member receiving slots 22. The tension can be maintained and the tissue 40 can be secured by placing the flexible member managing device 10 at rest on the patient's body, a covering on the patient, a surgical table under the patient, or a surface adjacent to the patient. In embodiments where an adhesive region 30 is employed or where the body 16 is made of a tacky medical grade silicone material, the flexible member managing device 10 can be adhered to the surgical site as detailed above. Moreover, the flexible member managing device 10 can easily be positioned and repositioned throughout the surgical procedure.

Next, the flexible members 12 can be removed from the flexible member receiving slots 22 as needed and the respective flexible members 12 are secured to the tissue 40. The user can switch between flexible members 12 to secure the tissue 40. After the respective flexible member 12 has been secured, the flexible member 12 can be placed back into its marked region on the extension by way of using the indicator 38.

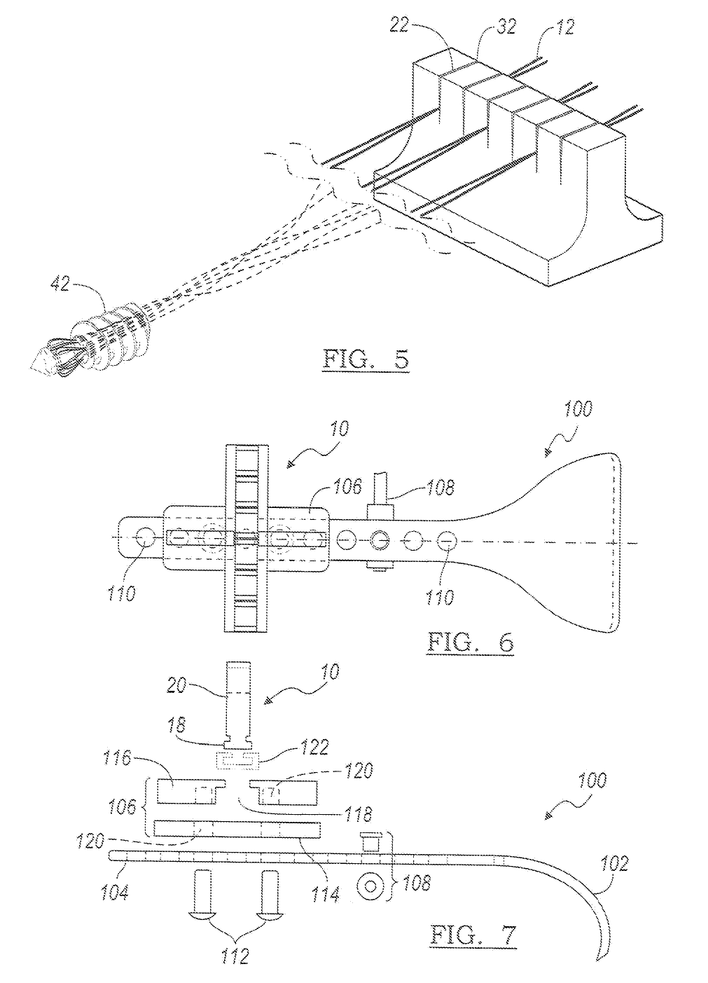

Turning to FIGS. 6 through 9, in various embodiments, the flexible member managing device 10 is disposed on a tissue retractor 100. The tissue retractor 100 includes a tissue engaging paddle 102 at its first or distal end and a handle 104 extending from the tissue engaging paddle 102. The flexible member managing device 10 is mounted at a selected position or station on the handle 104 using a sled 106. The tissue retractor 100 can be self-retaining end can utilize a supporting structure to maintain the tissue retractor 100 at the surgical site. An exemplary supporting structure is represented as a cross bar 108 which can be attached to another tissue retractor 100 at the surgical site. Generally, the tissue retractor 100 is made of surgical grade stainless steel. The tissue retractor 100 can also be made of any other biocompatible metal or non-metal, such as the metals and polymers listed above herein.

The tissue engaging paddle 102 defines a ledge to hook onto tissue at the surgical site. The ledge of the tissue engaging paddle 102 can be angled (90, 70, or 30 degrees as non-limiting examples), or it can be curved with respect to the handle 104.

The handle 104 defines a plurality of bores 110 which extend from near the paddle 102 to the proximal or second end of the tissue retractor 100. The bores 110 can be threaded, smooth, tapered, or have a uniform cross-section. The shape and characteristics of the bores 110 can be selected to match the corresponding fasteners 112. The bores 110 and the fasteners 112 facilitate the temporary securing of the sled 106 to the handle 104 for easy movement of the flexible member managing device 10 from a first location to a second location. The bores 110 provide a plurality of flexible member managing device stations along which the flexible member managing device 10 can be removably attached. The bores 110 can also provide an attachment point for the cross-bar 108.

The sled 106 includes a lower surface 114, an upper surface 116, an opening 118 defined by the upper surface, and at least one bore 120. The lower surface 114 can be placed adjacent to the handle 104. The at least one bore 120 can be aligned to directly communicate with at least one handle bore 110 such that the fastener 112 passes through the handle 104 and engages the sled 106. Although the sled 106 is depicted as being at a 90 degree angle with respect to the handle 104, it is understood that the sled 106 can be placed at any angle with respect to the handle 104 and can be rotated with respect to a position on the handle 104 intraoperatively. For example, where a single fastener 112 is used to secure the sled 106, the fastener 112 serves as a pivot point around which the sled 106 can be rotated to different angles.

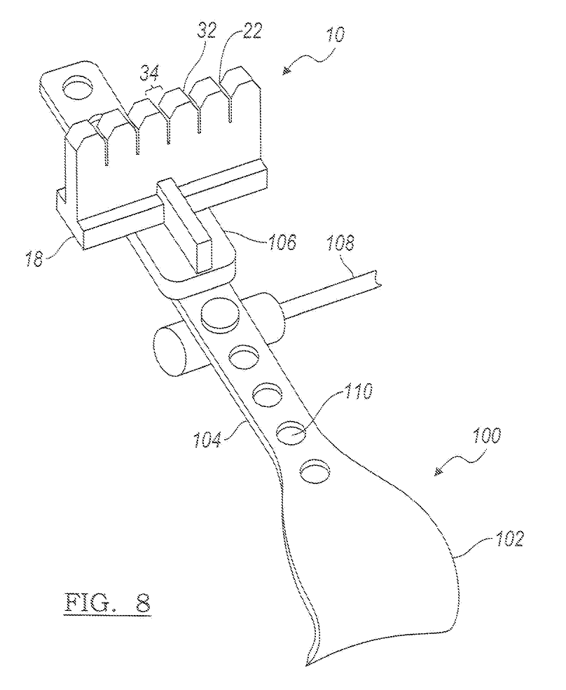

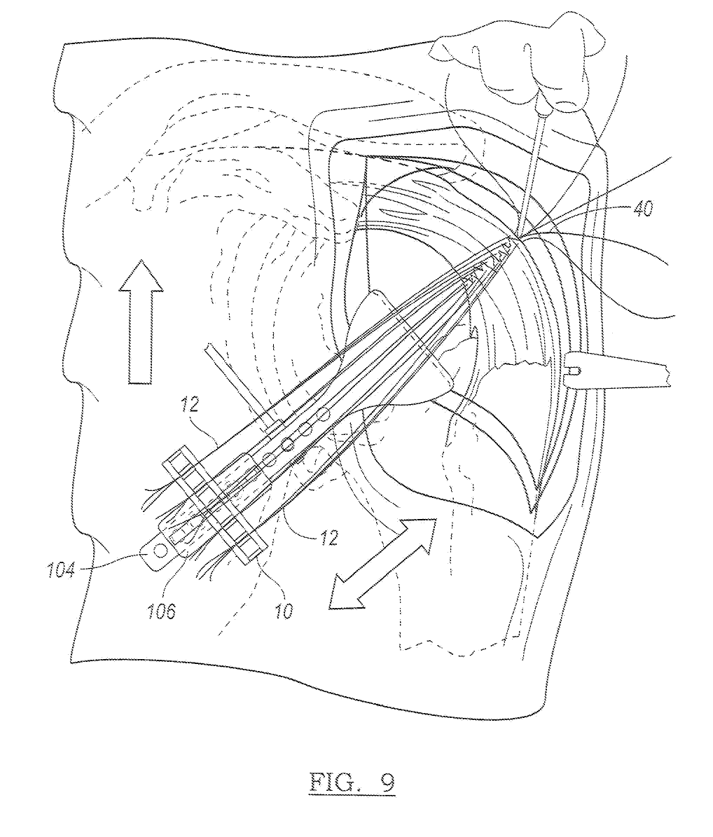

The upper surface 116 defines the opening 118 to accommodate the flexible member managing device 10. The opening 118 is sized to receive the flexible member managing device 10 by sliding, snapping, pressing, as non-limiting examples, the flexible member managing device 10 into the opening 118. In various embodiments and as shown in FIG. 8, the opening 118 can be generally rectangular to engage a portion of the base 18 of the flexible member managing device 10 depicted in FIG. 1A. The sled 106 and/or the opening 118 can also include curved regions to accommodate a flexible member managing device 10 such as those depicted in FIGS. 1C and 1E. It is understood that the sled 106 can have any suitable shape in which to fit the various flexible member managing devices 10 according to the present teachings.

Although the sled 106 is depicted as separate pieces in FIG. 7 to demonstrate the arrangement of the various components, it is understood that the sled 106 can be formed of a single piece or a plurality of pieces. The sled 106 can be made of stainless steel, or any other biocompatible metal or non-metal such as those listed above herein.

As best shown in FIG. 7, a cuff 122 can be incorporated into the opening 118 defined by the sled 106 to further maintain the flexible member managing device 10 in the sled 106. The cuff 122 can be of any suitable shape and can be adapted to fit within the sled opening 118. In various embodiments, the cuff 122 can be a part of the flexible member managing device 10, such as where the base 18 is a separate portion of the flexible member managing device 10 as best depicted in FIG. 1C. The cuff 122 can be sized to provide a snug fit in the opening 118 or the cuff 122 can be sized such that there is room to adjust the cuff 122 within the opening 118. For example, in an embodiment where the cuff 122 is smaller than the opening 118, the cuff 122 (and thereby the flexible member managing device 10) can be angled or rotated with respect to the surgical site. The angling and rotation can provide tension on the flexible members 12 to tighten tissue, as detailed herein. The cuff 122 can be made of the same material as the sled 106, the same material as the flexible member managing device 10, or the cuff 122 can be made of a different material. In various embodiments, the cuff 122 can be formed as an integral part of the flexible member managing device 10 and the cuff 122 and the flexible member managing device 10 can be used and then disposed.

To assemble the tissue retractor 100 and the flexible member managing device 10 combination, the flexible member managing device 10 is disposed in the cuff 122 and/or in the opening 118 on the sled 106. The sled lower surface 114 is then placed at a selected region on the handle 104 such that at least one handle bore 110 is in communication with at least one sled bore 120. At least one fastener 112 is then placed through the handle 104 and into the corresponding sled bore 120. The fastener 112 can be smooth or can be threaded or include another surface feature. The fastener 112 can also be tapered, wedge shaped, or have a uniform cross-section which is the same as or different from the cross-section of the at least one handle bore 110 and the at least one sled bore 120. The fastener 112 is engaged into the handle bore 110 to removably fix the tissue retractor 100. Exemplary engagement of the fastener 112 with the handle bore 110 and the sled bore 120 include press fit, interference fit, screw fit, and the like. It is understood that the various assembly stages can be rearranged or omitted (such as where the cuff 122 is or is not employed).

The present teachings also provide surgical procedures for use in orthopedic defect repair. The surgical procedures can be performed using one or a plurality of the flexible member managing devices 10 or the combination of tissue refractors 100 and flexible member managing devices 10 disclosed herein.

As shown in FIG. 5, the flexible member managing device 10 can be used to organize a plurality of flexible members 12 extending from an anchor 42, a graft, or other surgical implant. Anchors which incorporate a plurality of flexible members 12 and confine the flexible members at a single attachment point. As shown in FIG. 4, the flexible members 12 on the anchor 42 (not shown) extend from a centralized area of the surgical site 28, and the flexible member managing device 10 organizes the flexible members 12. Such organization is useful where each flexible member 12 may need to have a particular final placement respective to the anchor 42 and the tissue 40, such as with double row repair of a rotator cuff. As shown in FIG. 9, the flexible member managing device 10 is used to organize a plurality of flexible members 12 on the tissue retractor 100 used to secure the tissue 40.

Referring to FIG. 5, the flexible members 12 all extend from a proximal end of the anchor 42. The flexible member managing device 10 can be used such that each flexible member 12 need be identified once and then placed into the flexible member receiving slots 22 as to provide quick subsequent identification and selection. This increases the efficiency of the surgical procedure and is useful where several anchors 42 are employed. In certain rotator cuff repair procedures, six or more flexible members 12 can be used and there is a need for quick and proper identification of the flexible members 12. The quick and proper identification is also useful for minimally invasive procedures including orthopedic and non-orthopedic procedures.

Referring to FIG. 9, the tissue retractor 100 allows the flexible member managing device 10 to be temporarily fixed to the handle 104 and repeatedly moved with respect to the paddles 102 along a plurality of selected positions to extend the flexible member managing device 10 and the flexible members 12 away from the tissue 40. The benefits of efficiency from the placement, securing, and easy identification of the respective flexible members 12 are provided with the tissue retractor 100 and flexible member managing device 10 combination.

The description of the teachings is merely exemplar in nature and, thus, variations that do not depart from the gist of the teachings are intended to be within the scope of the teachings. Such variations are not to be regarded as a departure from the spirit and scope of the teachings.

What is claimed is:

1. A method of managing a plurality of flexible members at a surgical site comprising:
aligning at least one flexible member in at least one of a plurality of slots defined by a flexible member managing device to organize the plurality of flexible members; and
arranging the flexible member managing device remote from the surgical site.

2. The method of claim 1, wherein aligning the at least one flexible member in the at least one slot comprises removably fixing at least one flexible member in at least one slot.

3. The method of claim 1, wherein arranging the flexible member managing device comprises extending the device to a distance away from the surgical site to tension the flexible member on the flexible member managing device.

4. The method of claim 3, wherein the distance is sufficient to further tighten a tissue secured by the flexible member at the surgical site.

5. The method of claim 4, further comprising angling the flexible member managing device to provide tension on tissue at the surgical site.

6. The method of claim 1, further comprising identifying a flexible member by interpreting an indicator on the flexible member managing device.

7. The method of claim 1, further comprising arranging the flexible member managing device in a position selected from the group consisting of at an incline with respect to the surgical site, at a decreased elevation with respect to the surgical site, and leaned with respect to the surgical site.

8. The method of claim 1, wherein at least one of the plurality of flexible members is attached to an anchor.

9. The method of claim 1, further comprising securing the flexible member managing device to a surface in the immediate vicinity of the surgical site on a patient selected from the group consisting of: the patient, a covering on the patient, a surgical table under a patient, and a surface adjacent to the patient.

10. The method of claim 9, further comprising securing the flexible member managing device to a first position and repositioning the flexible member managing device to a second position; wherein the flexible members remain attached to the flexible member managing device throughout the positioning.

11. The method of claim 1, wherein the plurality of flexible members is simultaneously tensioned in relation to the surgical site.

12. The method of claim 1, further comprising moving the flexible member managing device through a range of motion while maintaining tension on the plurality of flexible members.

13. A method of managing a plurality of flexible members at a surgical site comprising:
attaching at least one of the plurality of flexible members to a rotator cuff;
aligning at least one flexible member in at least one of a plurality of slots defined by a flexible member managing device to organize the plurality of flexible members; and
arranging the flexible member managing device remote from the rotator cuff thereby tensioning the flexible member and effecting tissue closure of the rotator cuff.

14. The method of claim 13, further comprising securing the rotator cuff with a flexible member anchor wherein at least one of the plurality of flexible members is contained in the flexible member anchor.

15. The method of claim 13, further comprising simultaneously tightening the plurality of flexible members on the flexible member managing device.

16. The method of claim 13, further comprising disposing the flexible member managing device on an attachment region defined by a tissue retractor at the surgical site.

17. A flexible member managing device comprising:
a biocompatible body having a base;
an extension projecting from the base wherein at least a portion of the extension defines a plurality of flexible member-receiving slots to organize a plurality of flexible members; and
an attachment region operable to repeatedly fix the flexible member managing device intraoperatively.

18. The flexible member managing device of claim 17, wherein the flexible member receiving slots have a size selected from the group consisting of sized to removably secure the at least one flexible member and sized to grippingly engage the at least one flexible member.

19. The flexible member managing device of claim 17, wherein the flexible member receiving slots are tapered.

20. The flexible member managing device of claim 17, wherein the attachment region attaches the flexible member managing device near a surgical site.

21. The flexible member managing device of claim 20, wherein the attachment region comprises a plurality of flexible member managing device stations or an adhesive selected from at least one of: silicone, a suction cup, Velcro, or a biocompatible glue.

22. The flexible member managing device of claim 21, wherein the plurality of flexible member managing device stations are defined by a tissue retractor.

23. The flexible member managing device of claim 22, further comprising a sled to removably fix the base with respect to the tissue retractor.

24. The flexible member managing device of claim 22, further comprising a cuff to removably fix the base with respect to the tissue retractor.
